(19) United States
(12) Patent Application Publication
(10) Pub. No.: US 2026/0345419 A1
(43) Pub. Date: Nov. 5, 2026

(54) Method for Stabilized Movement of a Bipedal Exoskeleton

(71) Applicant: WANDERCRAFT, PARIS (FR)

(72) Inventors: Maxime BRUNET, PARIS (FR); Marine PETRIAUX, PARIS (FR)

(21) Appl. No.: 19/109,159

(22) PCT Filed: Sep. 5, 2023
(86) PCT No.: PCT/FR2023/051337

§ 371 Date: Mar. 6, 2025

(30) Foreign Application Priority Data
Sep. 6, 2022 (FR) 2208927

(51) Int. Cl. A61H 3/00; A61B 5/00; A61B 5/11

(52) U.S. Cl. CPC A61H 3/00; A61B 5/112; A61B 5/4836; A61B 5/7278; A61H 2201/5005; A61H 2201/5007

(57) ABSTRACT

Method for movement of an exoskeleton (1) receiving a human operator (2), for executing a step during which a first leg (30) of the exoskeleton (1) passes from a first control point to a second control point, and during which a second leg (31) of the exoskeleton (1) remains on the ground, the method comprising, at each iteration of a plurality of iterations implemented during the step, the following method steps: —estimation of a speed of the first leg (30); —determination of a trajectory of the centre of mass of the exoskeleton (1) guaranteeing the equilibrium of the exoskeleton (1), and of a speed-of-movement setpoint closest to the estimated speed; —based on the setpoint, determination of a first command to be applied to the first leg (30); and —based on the trajectory, determination of a second command to be applied to the second leg (31).

### FIELD OF THE INVENTION

The present invention concerns the field of exoskeleton robots. More precisely, it concerns a method for the stable movement of an exoskeleton.

### STATE OF THE ART

Recently, assisted walking devices called exoskeletons, which are external robotic devices that the operator (the human user) puts on by means of a system of fasteners that links the movements of the exoskeleton with the operator's own movements have emerged for people with significant mobility problems, such as paraplegics. Lower limb exoskeletons have several joints, usually at least at the knees and hips, to reproduce the walking movement. Actuators move these joints, which in turn move the operator. An interface system allows the operator and/or a physical therapist to give orders to the exoskeleton, and a control system transforms these orders into commands for actuators. Sensors are generally added to the device.

These exoskeletons are an advance over wheelchairs, as they allow operators to get back on their feet and walk. Moreover, exoskeletons are not limited by wheels and can theoretically be developed to operate in the majority of non-flat environments: Unlike legs, wheels do not allow overcoming major obstacles such as steps, stairs, obstacles of too great a height, etc.

In general, rehabilitation is carried out using the know-how of physical therapists. Various activities are practiced, to regain muscle in the limbs and especially to re-accustom the brain to giving electrical signals corresponding to coherent commands.

In this context, the exoskeleton can be used to guide patients' limbs in order to make them follow a predetermined trajectory during which the patient cooperates with the actuators of the exoskeleton and provides a part of the mechanical effort, making it possible to train lost bodily functions as described, for example, in international application WO 2022/053761 A1. The degree of effort sharing can be adjusted by the physical therapist, depending on the patient's abilities and the desired level of training.

However, unexpected deceleration or acceleration can cause the system to fall. Patients have difficulty making the movement at nominal speed, and the therapist must hold the exoskeleton to prevent it from falling. At nominal speed, the exoskeleton is balanced at all times. However, when the trajectory of the system is accelerated or decelerated, an inverse dynamic calculation reveals the instability of the exoskeleton.

This limitation has the consequence of preventing the assistance of the exoskeleton from freely adjusting to the efforts of the patient at all times. In addition, in the case of too severe deceleration, the physical therapist must exert excessive effort to support the exoskeleton.

One solution consists of developing a methodology for rapid replanning of a trajectory, as described, for example, in the document S. Caron and A. Kheddar, Multi-Contact Walking Pattern Generation Based on Model Preview Control of 3D COM Accelerations, Humanoids, pp. 550-557, 2016, Mexico. It is a flexible approach, able to deal with various experimental conditions and patient morphologies.

However, the replanning methodology may not find a balanced trajectory for certain exoskeleton velocities generated by the patient when exerting effort. Thus, the physical therapist cannot encourage the patient to make more effort during the movement to move faster and with less hesitation.

An object of the invention is to ensure the stability of the exoskeleton while guaranteeing freedom of movement to the patient.

### DISCLOSURE OF THE INVENTION

To this end, the invention provides a method for stabilized movement of a bipedal exoskeleton receiving a human operator to take a step during which a first leg of the exoskeleton passes from a first point of support on the ground to a second point of support on the ground, the first leg not being in contact with the ground between the first point of support and the second point of support, and during which a second leg of the exoskeleton remains in contact with the ground during the step;
the method comprising, at each iteration of a plurality of iterations implemented while taking the step, the performance of the following stages by a control unit on-board the exoskeleton:
   estimation of a speed of the first leg by a motion sensor connected to the control unit;
   from the estimated speed, determination of a movement speed setpoint and a trajectory of the exoskeleton center of mass;
the movement speed setpoint and the trajectory of the exoskeleton center of mass resulting from a resolution of an optimization problem having as objective that a difference between the movement speed setpoint and the estimated speed is minimized and as constraint that the trajectory guarantees the equilibrium of the exoskeleton until the first leg reaches the second support point on the ground with the speed of the movement speed setpoint;
   from the movement speed setpoint, determination of a first command to be applied to the first leg during the step;
   from the determined trajectory, determination of a second command to be applied to the second leg; and
   actuation of the first leg according to the first command and actuation of the second leg according to the second command.

The human operator can therefore set the first leg in motion, in position and speed, while the second leg remains on the ground in order to stabilize the exoskeleton on their support foot at each moment of the step. The first command is determined so that the movement speed setpoint is as close as possible to the estimated speed, without destabilizing the exoskeleton, and the second command is determined so that the exoskeleton can follow the trajectory of the center of mass predetermined.

In addition, a control unit on-board the exoskeleton can calculate the stabilizing trajectory of the center of mass over a sufficiently short period of time compared to a duration of one step so that a control loop of the control unit can generate, in near real time, an exoskeleton command that continuously adjusts to the patient's efforts.

The method allows responding to two major constraints, to best follow the speed of the patient's movements at each moment of the step and to respect the stability limits of the exoskeleton.

Thus, the invention makes it possible to use the exoskeleton for therapeutic purposes, more particularly in the context of rehabilitation of the lower limbs of the patient while ensuring the stability of the exoskeleton during the deceleration and acceleration phases of the patient's movements. Thus, the physical therapist no longer needs to hold the exoskeleton to prevent it from falling.

The plurality of iterations can be provided so as to move the exoskeleton from an initial position to a final position by a succession of steps.

It can be provided for the deviation to be zero when it is possible to determine a trajectory of the exoskeleton center of mass ensuring equilibrium of the exoskeleton until the first leg reaches the second ground support point with a movement speed setpoint equal to the estimated speed.

It can be provided for the movement speed setpoint to be determined from the estimated speed.

It can be provided for the determination, during an iteration, of the movement speed setpoint to be carried out by dichotomy over an interval comprised between the estimated speed and the movement speed setpoint determined during the preceding iteration.

It can be provided for the determination of the second command to comprise:
measuring the position of the center of mass of the exoskeleton and calculating a deviation by comparison of the measured position with the trajectory; and
from the calculated deviation, generating a position setpoint and a speed setpoint of one or more joints of the second leg.

It can also be provided for the generation of the position setpoint and the speed setpoint of one or each joint of the second leg to comprise the resolution of a hierarchical optimization problem whose maximum priority objective is to respect the position and speed of the first leg.

It can be provided for each iteration of the plurality of iterations to be repeated at a frequency of at least 250 Hz, preferably at least 500 Hz.

A bipedal exoskeleton comprising a control unit configured to implement the method according to the invention is also provided according to the invention.

The invention also provides a computer program product comprising instructions which, when the program is executed on a computer, cause the computer to implement the method according to the invention.

The invention further provides a computer-readable storage means on which the computer program product is stored.

### DESCRIPTION OF THE FIGURES

We will now present an embodiment of the invention by way of non-limiting example in support of the drawings in which:

FIG. 1 is a diagram of an exoskeleton in which the method according to the invention is implemented;

FIG. 2 is a diagram of an architecture for an implementation of the method according to the invention;

FIG. 3 is a flow chart illustrating the method according to the invention;

FIG. 4 is a graph showing a simulation result without the implementation of the method according to the invention, in which is illustrated the success rate corresponding to the number of times the exoskeleton has remained balanced as a function of the value of a movement speed setpoint of the first leg in relation to a nominal speed and the duration for which the movement speed setpoint is maintained;

FIG. 5 is a graph showing a simulation result with the implementation of the method according to the invention, in which is illustrated the success rate corresponding to the number of times the exoskeleton has remained balanced as a function of the value of a movement speed setpoint of the first leg in relation to a nominal speed and the duration for which the movement speed setpoint is maintained; and

FIG. 6 is a graph showing a simulation, during the exoskeleton step, of a variation around a nominal speed of the exoskeleton (dotted line), the entire set of movement speed setpoints of the exoskeleton guaranteeing its equilibrium (vertical bars) and the evolution of a movement speed setpoint determined by the implementation of the method according to the invention by the control unit (solid line).

### DETAILED DESCRIPTION OF THE INVENTION

### Architecture

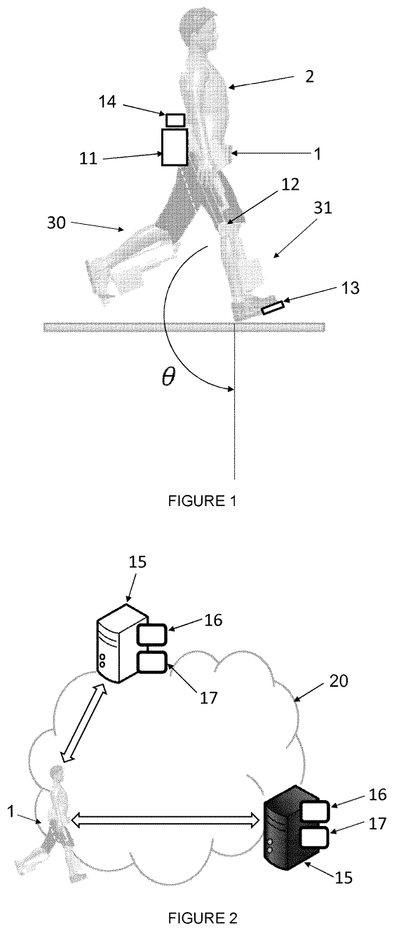

FIG. 1 and FIG. 2 illustrate a bipedal exoskeleton 1 capable of implementing the stabilized movement method according to the invention.

The exoskeleton 1 is an articulated mechanical system of the bipedal robotic device type, actuated and controlled, provided with a first leg 30 and a second leg 31, able to accommodate a human operator 2, each of whose lower limbs is joined to a leg of the exoskeleton 1 (in particular by means of straps). It can thus be a more or less humanoid robot. The term “walking” is understood here to mean the setting in motion of the robotic device 1, which translates in practice into an alternating support on the legs, in the standing position, so as to produce movement. A movement of the exoskeleton 1 is composed of a sequence of steps, each step having a foot of the first leg 30 lift off the ground and then rest again.

In the following, the term first leg 30 denotes a swing leg, the swing leg being the one that passes from a first support point on the ground to a second support point on the ground while not in contact with the ground between the first support point and the second support point, and the term second leg 31 denotes a support leg, the support leg being the one that remains in contact with the ground during the step.

For example, in FIG. 1, the operator 2 takes a step during which the swing leg 30 is lifted off the ground to swing forward while keeping the support leg 31 on the ground. In the case of a movement of the exoskeleton comprising a sequence of steps, the swing leg 30 and the support leg 31 alternate from one step to another.

The exoskeleton 1 has a plurality of degrees of freedom, i.e., hinges that are deformable (generally via rotation), i.e. movable relative to one another, each of which is either “actuated” or “non-actuated”.

For example, the exoskeleton 1 may comprise twelve actuated degrees of freedom and six non-actuated degrees of freedom.

An actuated degree of freedom refers to a joint provided with an actuator controlled by a control unit 11, i.e., this degree of freedom is controlled and can be acted upon. On the contrary, a non-actuated degree of freedom designates a joint without an actuator, i.e., this degree of freedom follows its own dynamics and the control unit 11 has no direct control over it (but in principal has indirect control via the other actuated degrees of freedom).

In the present case, the exoskeleton 1 has a variable level of assistance, i.e., the operator 2 can, by their movements, actuate the degrees of freedom by themselves insofar as the level of assistance allows, i.e., changing the orientation of a joint by themselves, for example flexing the knee. In this respect, the actuators are advantageously also sensors, in that they are capable of reporting their position to the control unit 11 so as to be able to detect the forced actuation of a degree of freedom by the operator 2.

Encoders 12, mounted on each joint of the exoskeleton 1, measure the position and the speed of each of these joints and make it possible to obtain a measured speed of the swing leg 30, for example according to an embodiment described in patent application FR 3, 113,829 A1.

The control unit 11 designates computer equipment, for example a processor, on board the exoskeleton 1 adapted to process instructions and generate commands intended for the various actuators. These actuators can be electric, hydraulic, etc.

This control unit 11 can also be connected to a remote server 15 comprising data processing means 16 and data storage means 17 in order to exchange information either intermittently or repeatedly during a step of the exoskeleton 1.

The control unit 11 is configured to implement the method according to the invention.

The present application will not be limited to any bipedal exoskeleton architecture 1, and the example described in applications WO 2015/140352 A1 and WO 2015/140353 A2 will be used.

Thus, preferably and in accordance with these applications, the exoskeleton 1 comprises on each leg a foot structure comprising a support plane on which a foot of the support leg 31 of the person wearing the exoskeleton can come to bear.

This support plane is substantially planar in shape and has, at one of its ends, a substantially curved edge such that the support plane constitutes an unactuated degree of freedom. It is also supplied with force sensors located on the support plane. The sensors also make it possible to obtain the torque of the external forces applied by the ground on the support plane.

However, the person skilled in the art will be able to adapt the present method to any other mechanical architecture.

In a known manner, the exoskeleton can be modeled in a simplified way by a linear inverted pendulum (LIP) model. With this model, the acceleration c of the center of mass is given by the following equation:

$$\ddot{c} = {ω}^{2} ( c - p )$$

with ω the pulsation in radians per second defined as the root of the ratio of the acceleration due to gravity to the vertical component of the position of the center of mass c;
c the position of the center of mass in meters; and
p the position of the center of pressure, the center of pressure corresponding to the barycenter of the reaction forces of the ground on the exoskeleton.

With this simplification, the method according to the invention can be executed repeatedly on the control unit 11 provided on the exoskeleton 1 during a step.

### Method for Stabilized Movement of the Bipedal Exoskeleton 1 Receiving the Human Operator 2 to Perform a Step

The present method cleverly dissociates the swing leg 30 from the support leg 31 of the exoskeleton 1 during a step set in motion by the operator 2 along a predefined trajectory, guaranteeing the stability of the exoskeleton during the step.

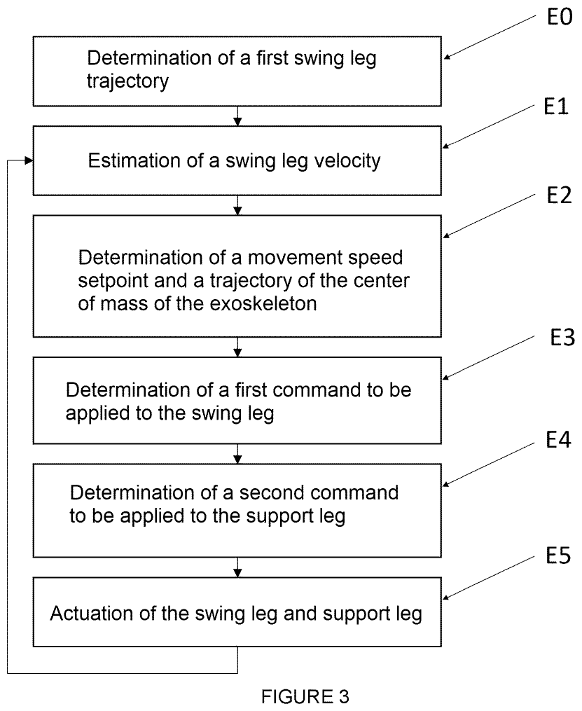

An embodiment of the method, illustrated in FIG. 3, is carried out according to the following stages.

During a first stage E0, the operator 2 takes a step during which a swing leg 30 of the exoskeleton passes from a first support point on the ground to a second support point on the ground, the swing leg 30 not being in contact with the ground between the first support point and the second support point, and during which a support leg 31 of the exoskeleton remains in contact with the ground during the step.

Thus, during the step, the swing leg 30 follows a first trajectory determined by the control unit 11 according to an embodiment described in patent application FR 3, 113,829 A1.

The trajectory of the swing leg 30 is derived from a stable nominal trajectory for both legs of the exoskeleton 1.

At each iteration of a plurality of iterations implemented during the step, the control unit 11 on board the exoskeleton 1 carries out the following stages.

During a stage E1, the encoders 12 connected to the control unit 11 estimate a speed Ve of the swing leg 30.

During a stage E2, the control unit 11 determines, from the estimated speed Ve, the movement speed setpoint Vd and a trajectory of the center of mass c of the exoskeleton 1, the movement speed setpoint Vd and the trajectory of the center of mass c of exoskeleton 1 resulting from a resolution of an optimization problem having as objective that a deviation between the movement speed setpoint Vd and the estimated speed Ve is minimized and as constraint that the trajectory guarantees the equilibrium of the exoskeleton 1 until the swing leg 30 reaches the second support point on the ground with the speed of the movement speed setpoint Vd.

The trajectory of the center of mass c is obtained by calculating the trajectory of the center of pressure p of the exoskeleton 1. Indeed, according to the inverted pendulum equation, for a given acceleration (corresponding, for example, to the tilting of the torso of the operator 2 forwards), the center of mass c is ahead of the center of pressure p so that they move away from each other. Thus, the center of pressure p can be chosen as a control variable enabling the exoskeleton 1 to be moved at the movement speed setpoint Vd.

The conditions to be verified by the trajectories to ensure the balance of the exoskeleton 11 until the swing leg 30 reaches the second ground support point are as follows:
C1: The trajectories of the center of mass c and the center of pressure p satisfy the equation ë=ω²(c−p), corresponding to a movement condition of the exoskeleton 1;
C2: The center of pressure p is included in a support polygon, corresponding to an equilibrium condition of the exoskeleton 1 during the movement;
the support polygon being defined as the convex envelope of all the points of contact of the exoskeleton 1 with the ground;
C3: The starting point of the trajectory of the center of mass c is identical to a current servo-control point of the center of mass c;
the servo-control point being determined by the previous iteration during the step; and
C4: The final point of the trajectory of the center of mass c is identical to the final point of the nominal trajectory of the center of mass c;
the nominal trajectory of the center of mass c is determined from the nominal articular trajectory by means of a kinematics calculation (stage E0).

Controlling the swing leg 30 at a movement speed setpoint Vd different from the estimated speed Ve causes the operator 2, when taking a step, to move more quickly or more slowly than they want, which has the effect of assisting the operator 2 and therefore of reducing the effects of rehabilitation. Indeed, when the exoskeleton assists the movement, the operator 2 is no longer in a case where they provide a force putting the swing leg 30 into motion.

Thus, the movement setpoint is determined from the estimated speed Ve and it is preferable for the movement speed setpoint Vd of the swing leg 30 to be as close as possible to the estimated speed Ve so as to minimize the effects of the control of the exoskeleton 1 on the swing leg 30 so as not to compromise the effect of rehabilitation.

According to one embodiment, the movement speed setpoint Vd and the trajectory are obtained by executing an on-line optimization, i.e., that is executed at each iteration of the plurality of iterations implemented during the step, for example by a quadratic programming (QP) dichotomy algorithm as follows:

A two-level optimization problem is formulated below. The higher level objective is the minimization of the difference between the movement speed setpoint Vd and the estimated speed Ve, the estimated speed Ve corresponding to the speed desired by the operator 2. The lower level constraint is the existence of a trajectory of center of mass c at the optimal speed of the upper level objective and which respects the conditions C1 to C4.

Let x⁰=x(t), x^{f} and T^{t} be the optimization parameters corresponding, respectively, to the current servo-control point of the center of mass c, to the end point of the nominal trajectory of the center of mass c and to an estimated time inversely proportional to the estimated speed Ve, find u* an optimal control of the center of mass c of the exoskeleton 1 and T* a time inversely proportional to the optimal speed, corresponding to the movement speed setpoint Vd closest to the estimated speed Ve and thus minimizing the deviation, such that

$${T}^{*} = {arg min}_{T ϵτ ( {x}^{0} , {x}^{f} )} \vert T - {T}^{t} \vert ,$$

$${u}^{*} = {arg min}_{u ϵΩ ( {x}^{0} , {x}^{f} , {T}^{*} )} {\int }_{0}^{{T}^{*}} {u}^{2} dt$$

with Ω all the commands and τ all the durations in which each element respects the conditions C1 to C4.

This optimization problem is indeed a bilevel optimization problem, i.e., the constraint of the higher level problem is itself the solution of an optimization problem. In this case, the lower-level optimization problem can be formulated as a quadratic problem.

The solution of this bilevel optimization problem, the search for the trajectory of the center of mass c, solution of the lower level problem whose movement speed setpoint Vd is closest to the estimated speed Ve and the determination, during the iteration of the plurality of iterations implemented during the step, of the movement speed setpoint Vd is carried out by dichotomy.

The dichotomy is performed over an initial interval between the speed determined at the previous iteration, for which the existence of a trajectory of the center of mass c respecting C1 to C4 is guaranteed by recurrence, and the estimated speed Ve, for which such an existence of a trajectory is not guaranteed. There is therefore always at least one trajectory in this initial interval corresponding to the trajectory of the previous iteration.

The initial interval is recursively replaced by several reduced intervals over a plurality of iterations as follows:
a mean is determined between a minimum value and a maximum value of a reduced interval;
a trajectory of the center of mass c and of the center of pressure p of the exoskeleton 1 are sought, the trajectory being traveled at a movement speed setpoint equal to the mean; and
the reduced interval is replaced by a first sub-interval between the bound of the reduced interval closest to the estimated speed Ve and the mean, if the trajectory respects conditions C1 to C4, otherwise the reduced interval is replaced by a second sub-interval between the mean and the bound of the reduced interval farthest from the estimated speed Ve.

The stages are then repeated over the reduced interval corresponding to the first sub-interval or the second sub-interval until the length of the reduced interval is small enough to obtain an acceptable estimate of the optimal speed.

Consequently, this method of resolution provides, at the same time, the optimal speed (according to the criterion of distance with the estimated speed Ve) and a trajectory of center of mass respecting C1 to C4 at this optimal speed.

This search method divides the search interval by 2 at each iteration of the dichotomy, guaranteeing algorithmic complexity of 2^{N} for the high-level objective, and therefore the speed of the method. The Applicant finds sufficient precision for the application using N=12.

If the optimal movement speed Vd found is equal to the estimated speed Ve, then the deviation to be minimized is zero and the optimization problem is solved from the first iteration of the dichotomy.

Otherwise, the dichotomy is continued until N=12 or until the length of the interval is reduced below a threshold D (typically of around 1e-3 to 1e-6).

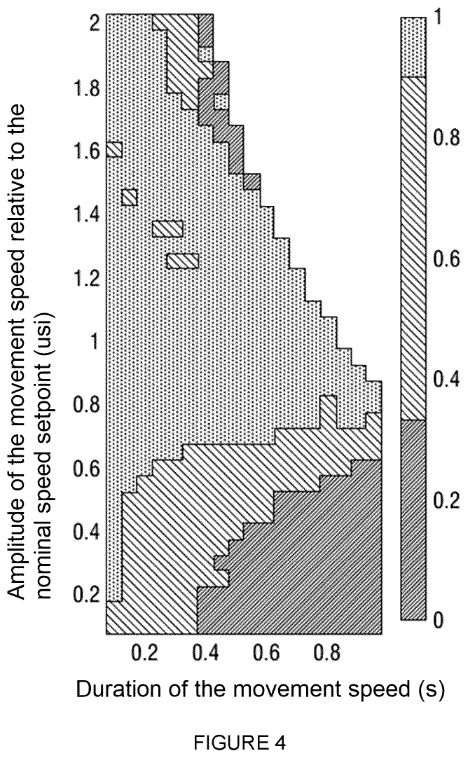

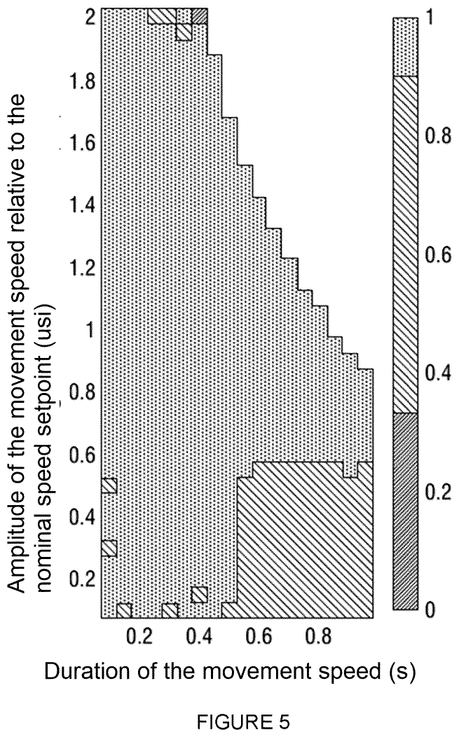

Such an embodiment makes it possible to maintain the balance of the exoskeleton for a wider range of speeds. Indeed, in comparison with FIG. 4, FIG. 5 shows that the method provides satisfactory exoskeleton stability (above 0.5) for a wider range of swing leg 30 movement speeds.

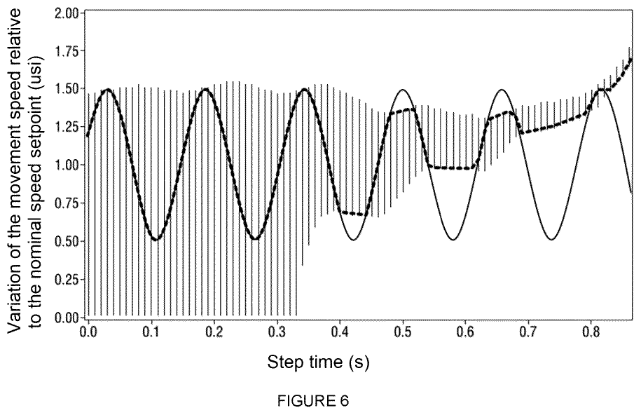

In particular, in order to guarantee the balance of the exoskeleton, the effect of the method is to increasingly constrain the speed requested by the patient as the swing leg 30 approaches the second support point. Indeed, in the case where an operator causes the swing leg 30 to accelerate and decelerate periodically throughout the step as illustrated in FIG. 6, the implementation of the method makes it possible to reduce the range of movement speeds (see the solid-line curve) in order to prioritize the balance of the exoskeleton 1 despite the movements of the operator 2 (see the dotted-line curve) when the swing leg 30 approaches the second point of contact corresponding to the end of the step.

The operator can therefore perform uncertain movements without the risk of falling. Thus, rehabilitation can be done as early as possible after an accident without the exoskeleton 1 being held by the physical therapist, allowing the operator 2 to recover the use of their limbs more quickly.

According to another embodiment, the set of trajectories guaranteeing balance can be generated off-line by exhaustive search for solutions to an optimization problem (for example, the optimization problem presented above), i.e., before the exoskeleton is set in motion by the patient, for a sufficiently large set of possible estimated speeds Ve, as well as a plurality of parameters, and this set can be recorded in the memory of the exoskeleton, possibly compressed by a learning model, for example a neural network. This set can then be evaluated on-line, therefore at each iteration of the plurality of iterations implemented during the step, to find the trajectory of center of mass c, possibly other auxiliary quantities which respect constraints C1 to C4 as well as possibly other constraints, and which minimize the difference between the movement speed Vd and the estimated speed Ve and possibly other criteria.

The neural network may, for example, be of the feedforward neural network (FNN) type.

The plurality of parameters useful for evaluating such a set of pre-calculated trajectories comprise the estimated speed Ve, a phase variable, i.e., a variable making it possible to estimate the advance of the swing leg 30 during the step, a current position of the center of mass around which the center of mass c of the exoskeleton 1 is controlled, and the final state of the center of mass obtained in stage E0.

This set of trajectories, optionally compressed by learning, is loaded by the control unit 11 from its internal storage space or by connecting to the server 15 via the network 20.

The control unit 11 can then use the neural network on each iteration of the plurality of iterations during the step to determine a trajectory guaranteeing the equilibrium of the exoskeleton 1 as a function of the estimated speed Ve.

Thus, a movement speed setpoint Vd as close as possible to the estimated speed Ve is calculated as a function of the trajectory generated by the neural network.

During a stage E3, the control unit 11 determines, from the movement speed setpoint Vd, a first command to be applied to the swing leg 30 during the step. The consequence of the first command is to modify the speed of the swing leg 30 and therefore the movement initially imposed by the user if the movement speed setpoint is different from the estimated speed.

During a step E4, the control unit 11 determines, from the determined trajectory, a second command to be applied to the support leg 31.

The second command to be applied to the support leg 31 has the effect of moving the center of pressure p in order to impose the desired trajectory on the center of mass c and thus to guarantee stability. The determination of the second command to be applied to the support leg 31 may be an admittance command comprising:
a measurement of the position of the center of mass of the exoskeleton 1 and a calculation of a deviation by comparing the measured position with the trajectory; and
on the basis of the calculated deviation, a generation of a position setpoint and a speed setpoint of one or more joints of the support leg 31.

The admittance command is described in detail in patent application FR 3, 117,393 A1.

The generation of the position setpoint and the speed setpoint of one or each joint of the swing leg 31 includes the resolution of a hierarchical optimization problem whose objective of maximum priority is to respect the position and speed of the swing leg 30.

The resolution of a hierarchical optimization problem consists of determining the position setpoint and the speed setpoint of one or each joint of the support leg 31 satisfying a list of objectives ranked by order of priority, the first objective of the list corresponding here to the objective of maximum priority.

An example of a list of ranked objectives is:
level 1, follow the position and the movement speed setpoint Vd of the swing leg 30;
level 2, follow the acceleration of the center of mass c of exoskeleton 1 as described in patent application FR 3,117,393 A1;
level 3, follow the roll and pitch movements of the pelvis; and
level 4, follow a joint configuration of the exoskeleton 1 standing and stopped during the step.

Thus, the operator 2 can carry out a rapid movement or a movement away from a direction of travel, for example a lateral movement, with the swing leg 30 without risking jeopardizing the stability of the center of mass.

During a stage E5, the control unit 11 actuates the swing leg 30 according to the first command and actuates the support leg 31 according to the second command.

Each iteration of the plurality of iterations is repeated at a frequency of at least 250 Hz, preferably at least 500 Hertz, e.g. 1 KHz.

The method described above can be repeated so that the plurality of iterations is repeated so as to move the exoskeleton 1 from an initial position to a final position by a succession of steps.

### Storage Medium and Computer Program Product

The invention also provides a computer program product comprising instructions which, when the program is executed on a computer, cause the computer to implement the method according to the invention.

The invention further provides a computer-readable storage means on which the computer program product is stored.

1. A method for stabilized movement of a bipedal exoskeleton receiving a human operator to take a step during which a first leg of the exoskeleton passes from a first point of support on the ground to a second point of support on the ground, the first leg not being in contact with the ground between the first point of support and the second point of support, and during which a second leg of the exoskeleton remains in contact with the ground during the step;
the method comprising, at each iteration of a plurality of iterations implemented while taking the step, executing by a control unit on-board the exoskeleton the stages of:
estimating a speed of the first leg by a motion sensor connected to the control unit;
based on the estimated speed, determining a movement speed setpoint and a trajectory of a center of mass of the exoskeleton;
said determining comprising solving an optimization problem having as objective that a difference between the movement speed setpoint and the estimated speed is minimized and as constraint that the trajectory guarantees the equilibrium of the exoskeleton until the first leg reaches the second support point on the ground with the speed of the movement speed setpoint;
based on the determined movement speed setpoint, determining a first command to be applied to the first leg during the step;
based on the determined trajectory, determining a second command to be applied to the second leg; and
actuating the first leg according to the first command and actuating the second leg according to the second command.

2. The method according to claim 1, wherein the plurality of iterations are repeated so as to move the exoskeleton from an initial position to a final position by a succession of steps.

3. The method according to claim 2, wherein determining, during an iteration, the movement speed setpoint is done by dichotomy over an interval comprised between the estimated speed and the movement speed setpoint determined during a preceding iteration.

4. The method according to claim 1, wherein determining the second command comprises:
measuring the position of the center of mass of the exoskeleton and calculating a deviation by comparison of the measured position with the trajectory; and
based on the calculated deviation, generating a position setpoint and a speed setpoint of one or more joints of the second leg.

5. The method according to claim 4, wherein generating the position setpoint and the speed setpoint of one or each joint of the second leg comprises solving a hierarchical optimization problem having as maximum priority objective respecting the position and speed of the first leg.

6. The method according to claim 1, wherein each iteration of the plurality of iterations is repeated at a frequency of at least 250 Hz.

7. A bipedal exoskeleton comprising a control unit configured to perform a method according to claim 1.

8. A non-transitory computer-readable medium storing a program including instructions that, when executed by a processor, cause the processor to implement the method according to claim 1.

9. (canceled)
